(19) United States
(12) Patent Application Publication
(10) Pub. No.: US 2021/0301347 A1
(43) Pub. Date: Sep. 30, 2021

(54) BIOMARKERS FOR EARLY EMBRYONIC VIABILITY AND METHODS THEREOF

(71) Applicant: UNIVERSITY OF TENNESSEE RESEARCH FOUNDATION, Knoxville, TN (US)

(72) Inventor: Ky G. Pohler, College Station, TX (US)

(21) Appl. No.: 17/342,112

(22) Filed: Jun. 8, 2021

Related U.S. Application Data
(63) Continuation of application No. 16/349,408, filed on May 13, 2019.
(60) Provisional application No. 62/420,670, filed on Nov. 11, 2016.

(51) Int. Cl. C12Q 1/6883; C12Q 1/6851; G01N 33/50

(52) U.S. Cl. CPC C12Q 1/6883; C12Q 1/6851; C12Q 2600/178; C12Q 2600/124; C12Q 2600/158; G01N 33/5076

(57) ABSTRACT

A method for determining early embryonic mortality (EM) in a female bovine includes determining an extracellular vesicle derived micro-ribonucleic acid expression profile of a serum (serum EV miRNA) obtained at from about 15 to about 30 days of gestation. The serum EV miRNA expression profile is compared to at least one reference serum EV miRNA expression profile to determine a serum EV miRNA expression profile indicative of early EM (EM EV miRNA expression profile). The EM EV miRNA expression profile may consist of an increased amount of at least one of miR-25, miR-16a/b, or miR-3596 compared to the at least one reference serum EV miRNA expression profile. Representative EM EV miRNA expression profiles and kits for determining EM EV miRNA expression profiles are provided.

This application is a continuation application of U.S. patent application Ser. No. 16/349,408 filed on May 13, 2019, which is the national stage of international patent application no. PCT/US2017/061306 filed on Nov. 13, 2017 which in turn claims priority from U.S. Provisional Patent Application Ser. No. 62/420,670 for Biomarkers for Early Embryonic Viability and Methods Thereof filed on Nov. 11, 2016, the entirety of the disclosures of which are incorporated herein by reference in their entirety.

### REFERENCE TO SEQUENCE LISTING SUBMITTED ELECTRONICALLY

A sequence listing electronically submitted with the present application as an ASCII text file named 1101-018rev2_ST25.txt, created on Nov. 2, 2020 and having a size of 3000 bytes, is incorporated herein by reference in its entirety.

### TECHNICAL FIELD

The present disclosure relates to markers of early embryonic mortality (EM). In particular, the present disclosure relates to the use of circulating extracellular vesicle (EV) micro-ribonucleic acids (miRNA) as markers for determination of EM.

### BACKGROUND OF THE INVENTION

About 85 to 95% of beef cattle exposed to a single artificial insemination (AI) undergo successful fertilization. However, there is a relatively high rate of early embryonic mortality (EM) in cattle and only about 60% of fertilized oocytes result in a pregnancy by day 30. In addition to this initial EM, there is a second phase of EM between days 30 to 45 of gestation, which corresponds to the time when the chorioallantoic cotyledonary placenta is forming in cattle. In cattle, real time ultrasonography can be used to diagnosis pregnancy throughout gestation starting as early as day 25. With the use of ultrasound and bovine pregnancy associated glycoproteins (PAGs), we have developed an animal model to investigate some of the mechanisms underlying late embryonic mortality (EM) between days 30 to 45 of gestation. However, these data from PAGs and ultrasound provide no information on early embryonic loss between fertilization and day 25 most likely due to limited methods to identify early embryonic loss. Additional markers such as IFN-stimulated gene (ISG) transcript abundance have been evaluated in leukocytes to study this earlier period of embryonic loss. However, since ISGs can be elevated in animals exposed to viruses (even when asymptomatic), the elevation of ISGs is not necessarily an indication that a viable conceptus is present but may be a good marker of nonpregnant animals.

In recent years, there has been a focus on microRNA (miRNA) as novel biomarkers. MiRNA are attractive biomarkers because they can be assayed in a non-invasive manner, are predictive, specific, sensitive and robust. The enhanced half-life of miRNA is thought to partially be a consequence of their prevalence in extracellular vesicles (EVs) of blood serum/plasma. Extracellular vesicles are comprised primarily of two major forms; exosomes that are intraluminal vesicles within multivesicular bodies and microvesicles that are derived from plasma membrane blebbing. Once released into bodily fluids, the origin (exosomal or microvesicle) is not easily determined thus EV is used to collectively describe these cell secreted vesicles. Evidence suggests that EV-derived miRNA can play specific rolls in cell to cell communication and overall biological function. Extracellular vesicle derived miRNA have become an attractive biomarker of many physiological and disease states. Specifically, in cancer screening and diagnostics, there have been over eight types of cancer (including lung, breast, and ovarian) that have specific EV-derived miRNA characterized as potential screening targets. In regards to reproduction, placental specific miRNA have been shown to be released and be detectable in the maternal circulation during pregnancy in women. Therefore, we tested the hypothesis that specific circulating EV-derived miRNA may differentiate pregnant versus EM and control cows during early (<30 day) gestation. The objective of this study was to determine if miRNA derived from EVs in the peripheral circulation were differentially abundant in pregnant vs EM cows at day 17 and 24 of gestation and to determine if these miRNA may provide reliable biomarkers for studying embryonic mortality or pregnancy maintenance.

### SUMMARY

In accordance with the foregoing need identified in the art, in one aspect methods are provided for determining early embryonic mortality (EM) in a female bovine is provided, comprising isolating serum from a blood sample obtained from the female bovine at from about 15 to about 30 days of gestation and determining an extracellular vesicle derived micro-ribonucleic acid expression profile of the serum (serum EV miRNA). The serum EV miRNA expression profile is compared to at least one reference serum EV miRNA expression profile. By this comparison, a serum EV miRNA expression profile indicative of early EM (EM EV miRNA expression profile) in the female bovine is determined.

In embodiments, the at least one reference serum EV miRNA expression profile is determined from one or both of: (i) a blood sample obtained from one or more reference pregnant female bovines at from about 15 to about 30 days of gestation, and (ii) a blood sample obtained from one or more reference non-pregnant female bovines. In other embodiments, the the at least one reference serum EV miRNA expression profile is determined from the blood sample obtained at from about 15 to about 30 days of gestation from the reference pregnant female bovine. The method further includes isolating EVs from the serum to provide a sample of isolated EVs and extracting ribonucleic acids from the isolated EVs. The EV miRNA may be amplified and quantified by high-throughput sequencing and reverse transcriptase quantitative PCR (RT-qPCR) to provide the serum EV miRNA profile and the at least one reference serum EV miRNA profile.

In embodiments, the EM EV miRNA expression profile consists of an increased amount of at least one of miR-25, miR-16a/b, or miR-3596 compared to the at least one reference serum EV miRNA expression profile. In other embodiments, the EM EV miRNA expression profile consists of an increased amount of miR-25, miR-16a/b, and miR-3596 compared to the at least one reference serum EV miRNA expression profile. In embodiments, the EV miRNA is amplified using primers selected from the group consisting of SEQ ID NO:9, SEQ ID NO:10, and SEQ ID NO:11. The method may include standardizing the EM EV miRNA expression profile and the at least one reference serum EV miRNA expression profile by normalizing effective miRNA read counts to a number of counts per million reads (cpm) and retaining only loci with a cpm greater than or equal to 10.

In another aspect, the present disclosure provides an extracellular vesicle derived micro-ribonucleic acid expression profile indicative of embryonic mortality (EM EV miRNA expression profile) at from about 15 to about 30 days of gestation in a female bovine, comprising an increased amount of miR-25, miR-16a/b, and miR-3596 in a serum EV miRNA expression profile of the female bovine compared to an amount of miR-25, miR-16a/b, and miR-3596 in at least one reference serum EV miRNA expression profile.

In embodiments, the amounts of miR-25, miR-16a/b, and miR-3596 are determined by high-throughput sequencing and reverse transcriptase quantitative PCR (RT-qPCR). In the expression profile, miR-25, miR-16a/b, and miR-3596 are amplified using primers selected from the group consisting of SEQ ID NO: 1, SEQ ID NO: 2, and SEQ ID NO: 3.

In embodiments, the at least one reference serum EV miRNA expression profile is obtained from one or both of: (i) a blood sample obtained from one or more reference pregnant female bovines at from about 15 to about 30 days of gestation, and (ii) a blood sample obtained from one or more reference non-pregnant female bovines. In other embodiments, the at least one reference serum EV miRNA expression profile is obtained from a blood sample obtained from one or more reference pregnant female bovines at from about 15 to about 30 days of gestation. The EM EV miRNA expression profile and the at least one reference serum EV miRNA expression profile may be standardized by normalizing to a number of effective miRNA read counts per million reads (cpm) and retaining only loci with a cpm greater than or equal to 10.

In yet another aspect of the disclosure, a kit for determining early embryonic mortality (EM) in a female bovine is provided, comprising a primer array comprising primer sequences for determining expression levels of one or more extracellular vesicle micro-ribonucleic acids (EV miRNAs) indicative of said EM. Optionally, the kit may include reagents for extracting the EV miRNAs and reagents for performing reverse transcriptase quantitative PCR (RT-qPCR) of the extracted EV miRNA.

In embodiments, the kit primer array comprises primer sequences for determining expression levels of one or more of miR-25, miR-16a/b, and miR-3596 extracted from an extracellular vesicle micro-RNA (EV miRNA). The primer array may include the primer sequences set forth as SEQ ID NO:1, SEQ ID NO:2, and SEQ ID NO:3. In embodiments, the primer array consists of the primer sequences set forth as SEQ ID NO:1, SEQ ID NO:2, and SEQ ID NO:3.

In the following description, there are shown and described embodiments of the disclosed methods and kits for selecting for determining early EM. As it should be realized, the methods and kits are capable of other, different embodiments and their several details are capable of modification in various, obvious aspects all without departing from the devices and methods as set forth and described in the following claims. Accordingly, the drawings and descriptions should be regarded as illustrative in nature and not as restrictive.

### BRIEF DESCRIPTION OF THE DRAWINGS

The accompanying drawing figures incorporated herein and forming a part of the specification illustrate several aspects of the disclosure, and together with the description serve to explain certain principles thereof. In the drawings:

FIG. 1 illustrates the experimental design for the present studies;

FIG. 2A shows gel electrophoresis showing PCR products of ISG-15, on day 0 and 17 of gestation, in a cow that was pregnant or experienced embryonic mortality;

FIG. 2B shows a Western blot showing presence of CD81, a well characterized EV marker, in serum derived EVs from a cow assigned to the pregnant group. A similar band was detected in all EV samples from the pregnant, embryonic mortality and control groups;

FIG. 3A shows profiles of small RNAs that were extracted from EVs obtained from a pregnant cow;

FIG. 3B shows profiles of small RNAs that were extracted from EVs obtained from an Embryonic Mortality cow;

FIG. 3C shows profiles of small RNAs that were extracted from EVs obtained from a Control cow;

FIG. 4 shows relative percentage of small RNAs were obtained from circulating EVs;

FIG. 5 shows Day 17 EV-derived circulating microRNA across all treatment groups;

FIG. 6 illustrates detection of known EV-derived miRNA by RT-qPCR in pregnant cows and cows experiencing embryonic mortality. A total of 4 miRNAs were tested using RT-qPCR. At d17 of gestation, 3 miRNAs (miRNA 16b, 25, and 3596) were confirmed with RT-qPCR. miR-100 was not validated based on RT-qPCR (data not shown).

FIG. 7A illustrates biological function analysis for 29 known miRNA that were differentially abundant between the EM and pregnant groups, with significant upregulation of 12 functions on d 17 of gestation of the differentially abundant miRNA between the EM and pregnant groups; and

FIG. 7B illustrates biological function analysis for 29 known miRNA that were differentially abundant between the EM and pregnant groups, resulting in significant hit on the targets PTGS2, SLC38A1, IGF-1R, Akt, TRMT1, SNAI1, Cg, CAMTA1, MAP2K1/2, BCL6 and TP53.

Reference will now be made in detail to embodiments of the disclosed subject matter, examples of which are illustrated in the accompanying drawing figures.

### DETAILED DESCRIPTION

Any citations, gene sequences, accession numbers, and reference sequences included or referred to in this application form a part of the disclosure and are incorporated herein in their entirety by reference. It will be appreciated that the embodiments shown and described in this patent application are an illustration of one of the modes best suited to carry out the invention. The invention is capable of other different embodiments, and its several details are capable of modification in various, obvious aspects all without departing from the invention. Accordingly, the drawings and descriptions provided herein will be regarded as illustrative in nature and not as restrictive. Various embodiments of the methods and compositions of the present disclosure will now be described by way of the following Examples.

### Materials and Methods

Treatments: All experimental procedures were approved by University of Missouri Institutional Animal and Care and Use Committee (ACUC protocol 8444). Estrous cycles of postpartum suckled beef cows (n=36) from the University of Missouri Beef Research and Teaching Farm were synchronized with the 7 day CO-synch CIDR synchronization protocol (GnRH (100 μg as 2 mL i.m. of Cystorelin, Merial and an Eazi-Breed CIDR insert [1.38 g progesterone; Zoetis]) on day −9, prostaglandin F2α [PG; 25 mg as 5 mL i.m. of Lutalyse, Pfizer Animal Health] and CIDR removal on day −2, and a second injection of GnRH [100 μg as 2 mL i.m. of Cystorelin, Merial] 66 h after PG) and AI on day 0. All cows were inseminated to the same sire, 36 cows were inseminated with live sperm and 8 control cows were inseminated with dead sperm. Control cows received dead semen (motility was no longer present) from the same sire to ensure that all animals were exposed to the same seminal plasma and sperm, since both are known to contain miRNA. On day 17, the control cows also received a CIDR to maintain a similar level of progesterone as the cows that established pregnancy from day 17 to 30 (pregnancy diagnosis). Those cows in the live sperm AI group were subsequently divided into two groups following the day 30 pregnancy diagnosis: pregnancy establishment and maintenance to day 30 (pregnant group; n=17) and pregnancy establishment but not maintenance to day 30 (embryonic mortality group, EM; n=19). Embryo mortality was distinguished from failure to conceive by detection of increased expression of ISGs (ISG15, Mx2 and OAS1; Green et al., 2010) on day 17 compared to day 0, but no embryo present on day 30. For example, if ISG transcripts were more abundant at day 17 compared to day 0 (evidence of IFN-tau production by an embryo) and an embryo was not detected on day 30, animals were considered to have lost an embryo and were assigned to the “embryo mortality (EM) group.” In comparison, if ISG transcripts were more abundant at day 17 compared to day 0 and an embryo was detected on day 30, cows were determined to have conceived and maintained pregnancy by transrectal ultrasound and visualization of a fetal heartbeat (assigned to the ‘pregnant group’). In addition, control cows, included in the experiment, exhibited either a decrease or no change in ISG transcript abundance from day 0 to 17 of gestation. The control group was included to compare EV miRNA in circulation of cows that could not have conceived with cows that established and maintained (pregnant group) or did not maintain a pregnancy (EM group).

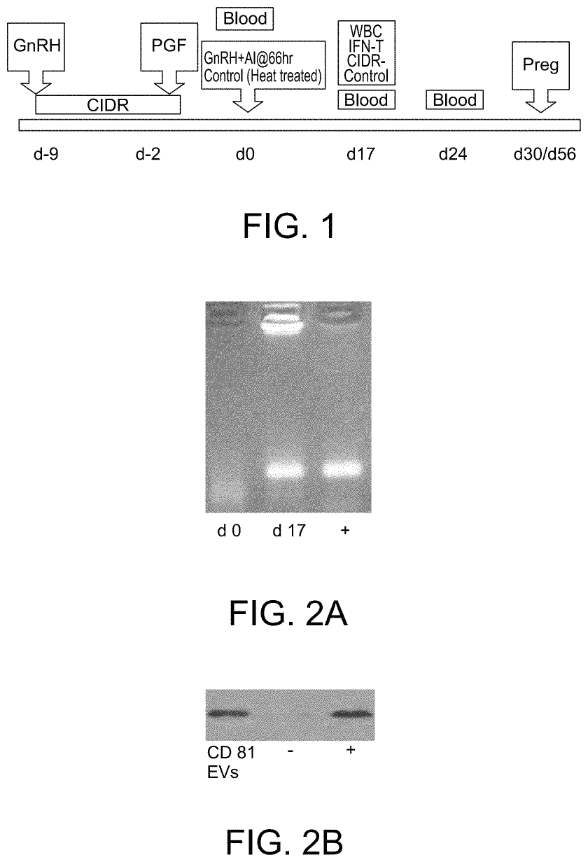

FIG. 1 illustrates the estrus synchronization protocol and sample collection schedule used in this experiment. Lactating beef cows (n=44) were synchronized with the Co-synch+CIDR protocol: administration of gonadotropin releasing hormone (GnRH) and CIDR on day −9, prostaglandin F_{2α} (PGF) on day −2, and Fixed timed AI and GnRH on day 0. Cows were divided into two groups those artificially inseminated (AI) with live sperm (n=36) with remainder (n=8) receiving dead sperm. Control cows received a CIDR from day 17 until day 24 of gestation to maintain elevated circulating concentrations of progesterone. Blood was collected on day 0, 17, and 24. Interferon stimulated gene activity (IFN-T) was evaluated on day 0 and d 17. Pregnancy status was determined by transrectal ultrasound on day 30 and 56 of gestation.

Blood Collections: All cows were bled at the time of AI (day 0), day 17 and day 24. Blood serum and plasma samples were harvested by venipuncture into a 10-ml vacutainer tube (BD Vacutainer, Becton, Dickinson and Company, New Jersey) and a 10-ml EDTA treated vacutainer tube (BD Vacutainer, Becton, Dickinson and Company, New Jersey), respectively. The serum tube was allowed to clot at room temperature for 1 hour before being placed in a 4° C. refrigerator for approximately 24 hours. Following centrifugation, serum was collected and stored at −80° C. until measurement of progesterone on day 0, 17 and 24 or EV extraction on day 17 and 24. The plasma sample was immediately placed on ice, where it remained until centrifugation. Plasma was decanted and white blood cells collected by buffy coat extraction as explained by Stevenson et al. (2007). White blood cells were harvested on days 0 and 17.

White blood cell RNA extraction and cDNA synthesis: White blood cell buffy coats were extracted for RNA using Trizol reagent (Life Technologies) according to the manufacturer's recommendations. Complementary DNA was synthesized from 2 μg of RNA using the PrimeScript™ First Strand cDNA Synthesis Kit (Catalog number: 6110A; Lot number: AK3101) from Takara Bio Inc based on manufacturer's recommendations.

PCR for interferon-stimulated gene (ISG) expression: Leukocyte RNA was prepared for PCR with AccuPrime™ Taq. 2.5 μl of AccuPrime™ Buffer, 0.5 μl of 60 ng/μl forward and reverse primers (see Table 1 for ISG15, MX2 and OAS1 and RPL7) 0.5 μl AccuPrime™ Taq DNA Polymerase, 21 μl of water, and 0.5 μl of cDNA as previously described by Green et al., 2010. PCR reactions occurred at 95° C. for 2 minutes, 40 cycles of 95° C. for 30 seconds, 54° C. for 20 seconds, and 68° C. for 4 minutes. Samples were then cooled to 4° C. PCR products were separated on a 1% agarose gel containing 1 μg/ml ethidium bromide in order to visualize the products. Interferon-stimulated gene expression (ISG15, MX2 and OAS1) was then determined by the presence or absence of a product band on the gel. RPL7 was used as a positive control for all samples.

Extracellular Vesicle Isolation: Isolation of EVs from 2 mL of serum of each cow on day 17 and 24 followed a modified protocol that had been previously validated for collection of EVs [24, 25]. Samples were handled individually and never pooled. Each 2 mL sample was centrifuged for 10 minutes at 300×g to remove any cellular debris. Cleared supernatant was added to ultra-centrifuge tubes (Beckman Coulter 347357) and an additional 2.5 mL of phosphate buffered saline (PBS) was added to each sample. Samples were subsequently spun at 4° C. for 20 minutes at 2000×g. Again supernatant was transferred into fresh ultra-centrifuge tubes and spun at 18,000×g for 45 minutes at 4° C. Following this spin, the supernatant was filtered through a Millex GP 0.22 um filter with a 5 mL insulin syringe into a new ultra-centrifuge tube. The filter was rinsed with PBS and the tubes were balanced with PBS. Samples were centrifuged at 110,000×g for 3 hours at 4° C. Following the 3 hour spin, a visible white pellet could be seen for each of the samples in the tubes. Pellets were rinsed with PBS and spun for an additional 90 mins at 110,000×g. Pellets were suspended in 50 ul of PBS and 5 ul was saved for western blot analysis and nanoparticle tracking while the remaining 45 ul was used for RNA extraction as described below.

Western Blot Analysis: Purified EVs were suspended in 40 μl of M-PER (Thermo Scientific) with HALT protease inhibitor cocktail (Thermo Scientific) for 15 minutes on a tube rotator at room temperature. Lysates were mixed with Laemmli sample buffer (31.5 mM Tris-HCl, pH 6.8; 10% glycerol; 5% β-mercaptoethanol; 1% SDS; 0.01% bromophenol blue), denatured at 95° C. for 5 minutes, and separated by SDS-PAGE at a constant voltage of 150 V for approximately 60 minutes in 1× running buffer (25 mM Tris, 192 mM glycine, 0.1% SDS). Protein was transferred to 0.45 μm Protran BA 85 nitrocellulose membrane (GE Healthcare, Buckinghamshire, UK) in Towbin transfer buffer (25 mM Tris, 192 mM glycine, 20% methanol) at 100 V for 60 minutes. Membranes were placed in blocking buffer (TBS, 5% non-fat milk, 0.1% Tween 20) for 1 hour at room temperature. Primary antibodies [CD81; Santa Cruz Biotechnology; [26]] were diluted 1:20,000 in blocking buffer and incubated with the blots for 1 hour at 4° C. Membranes were washed with TBS containing 0.1% Tween 20 (TBST) before incubation with goat anti-rabbit HRP conjugated secondary antibody at 1:10,000 dilution for 1 hour at room temperature. Membranes were washed with excess TBST and incubated with SuperSignal West Pico Chemiluminescent Substrate (Thermo Scientific) for 3 minutes prior to imaging with a ChemiDoc MP system and Image Lab 4.1 software (BioRad, Hercules, Calif.).

Nanoparticle Tracking Analysis: Quantification of nanoparticles (EVs) was conducted similar to the method reported by Navakanitworakul et al., [27]. Briefly, all nanoparticle quantification was performed on a NanoSight LM-10HS (Malvern Instruments Ltd, Worcestershire, UK). Prior to quantification, aliquots were diluted to approximately 1-8×10⁸ per millimeter to conduct the analysis. For quantification purposes, 3 videos were recorded for 60 seconds and subsequently analyzed using the Nanosight NTA 2.4 software (Malvern Instruments Ltd, Worcestershire, UK). All samples were quantified in replicates of three. Data was then analyzed using SAS 9.4 PROC GLM package.

MiRNA extraction: Extraction of RNA was performed with Trizol reagent based on the manufacturer's recommendations. In order to determine quantity and quality of small RNAs, samples were evaluated on a small RNA Labchip kit (Agilent Technologies) by using the Agilent 2100 Bioanalyzer, according to the manufacture's recommendations.

MiRNA sequencing: All miRNA sequencing was performed on the Illumina HiSeq2500 system at the University of Kansas Medical Center—Genomics Core (Kansas City, Kans.). Extracellular vesicle RNA (ranging from 1.8 ng-100 ng) was used to initiate the TruSeq Small RNA library preparation protocol (Illumina #RS200-0012 kit A). The EV RNA was ligated with 3′ and 5′ RNA adapters followed by a modified reverse transcription reaction and modified PCR amplification. Due to low starting quantities of the EV RNA, the reversed transcription of the RNA adapter ligated samples was modified by performing two duplicate reactions containing 60 of the 3′/5′ RNA ligated RNA. The 12.50 yield of each duplicate reverse transcription reaction was pooled to obtain 250 of homogeneous cDNA. The subsequent PCR amplification, with index adapter incorporation, was modified by replacing 8.50 of ultra-pure water in the PCR master mix with 8.50 of the reverse transcribed and pooled cDNA (210 cDNA total). The modified PCR reaction was performed with 15 cycles of amplification.

Size selection and purification of the cDNA libraries were conducted using 3% marker H gel cassettes on the Pippin Prep size fractionation system (Sage Science). The Agilent 2100 Bioanalyzer was used with the High Sensitivity DNA kit (Agilent #5067-4626) or the DNA1000 kit (Agilent #5067-1504) to validate the purified libraries.

Libraries were quantified on the Illumina ECO Real Time PCR System using KAPA SYBR Universal Library Quant kit—Illumina (KAPA Biosystems KK4824). Following quantification, libraries were adjusted to a 2 nM concentration and pooled for multiplexed sequencing. Libraries were denatured and diluted to the appropriate pM concentration (based on qPCR results) followed by clonal clustering onto the sequencing flow cellusing the TruSeq Rapid Single Read (SR) Cluster Kit-HS (Illumina GD402-4001). The clonal clustering procedure was performed using the automated Illumina cBOT Cluster Station. The clustered flow cell was sequenced on the Illumina HiSeq 2500 Sequencing System in Rapid Read mode with a 1×50 cycle read and index read using the TruSeq Rapid SBS kit-HS (Illumina FC402-4002). Sequencing was performed to obtain an unbiased global profile of small RNA in the three groups (Control, EM and Pregnant) at the two time-points (day 17 and day 24). The groups were analyzed in biological quadruplicates giving 24 samples in total. High throughput sequencing was done at a 50 bp single-end resolution. Following collection, sequence data was converted from .bcl file format to FASTQ files and sorted based on the particular index sequence present for further downstream analysis.

Small RNA Processing: The mapping and identification of known and novel miRNA strategy used was previously described in detail [27]. After 3′ adapter removal, the sequencing reads were mapped using the Bowtie2 software [28], in the local sensitive mode, to the bovine genome (assembly UMD3.1). The mapped reads were further processed as follows. The reads from all 24 samples were merged and scanned for high-density regions defined as a contiguous region whose read count at each base is not less than 20% of the highest base read count for the locus. These high-density regions formed the effective region of the locus and its length was its effective length. Loci with an effective length greater than or equal to 18 were retained. The number of reads mapped to the effective region in each sample formed the effective read counts. Loci were further filtered on their normalized effective read counts (normalized to the number of counts per million reads (cpm)), retaining only those loci with a cpm greater than or equal to 10 in all 4 replicate samples in at least one of the six biological conditions. The distribution of these loci (miRNA) across the different biological conditions is shown in Table 2. These loci were used for down-stream analysis. The effective regions were annotated for genomic features from the Ensemble gene annotation file for bovine (release 70) and miRBase (release 21).

Effective regions that mapped to annotated bovine mature miRNA were first identified, the remaining effective regions were compared to known miRNA from both bovine and other species found in miRBase (release 21). A region was labeled as a miRNA by homology if it passed the following criteria; a gapless alignment of the effective region to the mature reference miRNA with at most 2 mismatches in the core and at most 1 gap/mismatch at the 5 and 3 prime ends and less than 10% mismatches in the alignment of the reference hair-pin sequence to the extended locus region in the genome. Novel computationally identified miRNA were validated based on the criteria that the extended effective region should have a predicted pre-miRNA like hairpin structure [29] with the effective region falling in the stem region with at least 80% pairing [27].

Generalized linear models (GLM) developed for multi-group experiments available from the edgeR software package [30] were used to determine significantly differently expressed miRNA between the different conditions. For differential expression analysis, miRNA had to have a cpm greater than or equal to 10 in all 4 replicate samples in at least one of the two groups being compared. The edgeR package employs advance empirical Bayes methods to estimate miRNA-specific biological variation under minimal levels of biological replication. The RNA composition in each sample was normalized in edgeR using the trimmed mean of M-values (TMM) method. The associated p-values were corrected for multiple-hypothesis testing (FDR) by the Benjamini and Hochberg method [31]. Absolute expression differences greater than or equal to 1.5 with a FDR less than or equal to 0.1 were considered significant.

Biological Functional Analysis: The biological functions associated with differentially expressed miRNA in day 17 pregnant versus EM samples were obtained using Ingenuity Systems IPA (ingenuity.com) software. IPA consists of a comprehensive knowledge base of known molecular interactions, including miRNA. Using this information, IPA computes an enrichment score for different biological functions based on the uploaded genes. Enriched biological functions for a set of miRNA is inferred by the p-value of the measure of likelihood of the overlap of target miRNA and the genes in the relevant biological function calculated using the right tailed Fisher's exact test. Biological functions with an associated p-value less than or equal to 0.05 were considered enriched for the target miRNA. Since IPA's knowledge base is confined to information on gene and gene products on human, mouse or rat, the bovine miRNA references were converted to their best matched human or mouse homolog before uploading to IPA.

Quantitative PCR: Small RNA isolated as described above and previously used for Illumina sequencing, was polyadenylated, followed by cDNA synthesis using a poly-T primer with a 3′ degenerate anchor and a 5′ universal tag (Exiqon miRCURY LNA™ System) according to manufacturer's recommendations. The cDNA template was then amplified using miRNA-specific (see Table 1) and LNA™-enhanced forward and reverse primers using SYBR Green detection in an ABI 7300 real time PCR machine, Exiqon LNA miRNA primer sets were used to amplify miRNA sequences (Catalog #204306, 2114063, 204361, 206037). PCR reactions without template were used as negative controls. Threshold measurements were set in the linear region of the amplification plot above the baseline and quantification cycle (Ct) were determined based on the cycle number in which the threshold line intersected the amplification line. The LNA specific control primers U6 snRNA (#203907) and SNORD 49A (#203904) were used as the reference genes in all reactions for data normalization. Abundance values were calculated using the mean (2^{-ΔΔCt}) of the U6 and SNORD 49A references in the control samples, which were considered to be baseline and the mean of the target miRNA gene Ct values.

TABLE 1
Primer sequence (5′ to 3′) of certain miRNA
amplified during PCR and qPCR.

| miRNA | Primer Sequence |
|---|---|
| miR- | 5′-UAGCAGCACGUAAAUAUUGGC-3′ |
| 16a/b | (SEQ ID NO: 1) |
| miR-25 | 5′-CAUUGCACUUGUCUCGGUCUGA-3′ |
| | (SEQ ID NO: 2) |
| miR- | 5′-AACCACACAACCUACUACCUCA-3′ |
| 3596 | (SEQ ID NO: 3) |

Assays: Serum progesterone concentrations were quantified by RIA with a Coat-a-Count RIA kit (Diagnostic Products Corporation, Los Angeles, Calif.) as described previously [32]. Intra assay coefficients of variations were 4.82% and the assay sensitivity was 0.08 ng/mL for the progesterone RIA. Serum concentrations were analyzed using SAS 9.4 PROC GLM package.

### Results

IFN-stimulated gene (ISG) expression in leukocytes was measured in all animals on day 0 and 17 of gestation (FIG. 2). These qualitative measurements of ISG15, Mx2 and OAS1 were performed to assign the inseminated animals to the appropriate experimental groups. Animals must have had an increase in at least 2 of the 3 specific ISGs to be considered pregnant on day 17. Based on PCR and gel electrophoresis cows assigned to the pregnant and embryonic morality groups had low and increased IFN-t stimulated gene expression on day 0 and day 17 of gestation, respectively; whereas, control cows had nondetectable or low IFN-t stimulated gene expression on both days. (+ sign indicates a positive control sample). All animals submitted for deep sequencing had positive serum EV immunoreactivity for CD81, a well-characterized EV protein marker, at both day 17 and 24 of the study (FIG. 2). Based on nanosight particle analysis, the overall mean diameter of EVs was 109 nm±42 (mean±SD) for all animals sequenced. Overall mean number of particles across all samples was 7.3×10⁷±1.07×10⁷/mL of serum (mean±SD). There were no significant differences detected among the groups in total number of particles counted or size of particles for a specific group. There was also no difference in circulating concentrations of progesterone on day 17 or day 24 among the pregnant, EM and control (day 17 CIDR-implanted) animals.

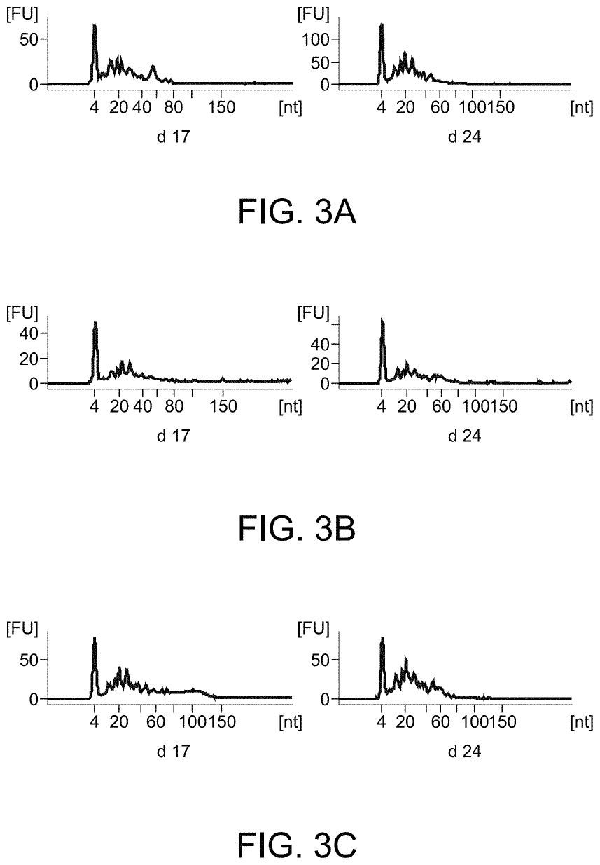

The small RNA profiles of purified EVs from pregnant, EM and control groups were collected using a small RNA Labchip on an Agilent 2100 Bioanalyzer (FIG. 3). The y axis represents the amount [FU] and the x axis represents the base pair size [nt]. Note the clustering of RNA species below 60 base pairs in all groups of cows demonstrating the increased abundance of small RNAs. These data revealed that circulating EVs contained mostly small RNA species less than 60 bp in length across all groups. These results also suggest that the extracted EVs contained other small RNAs other than just miRNA.

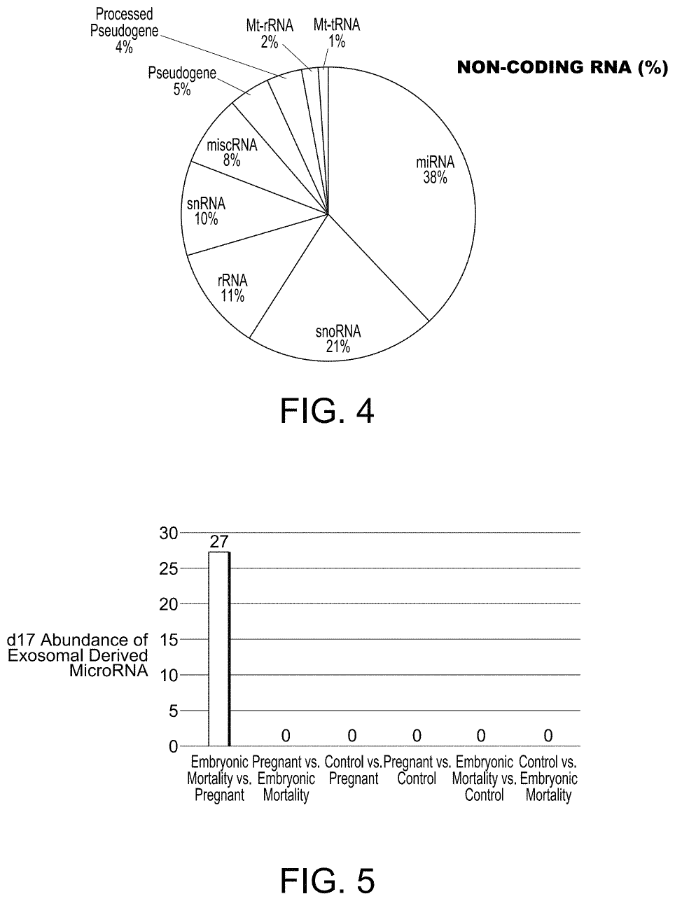

Small RNA sequencing confirmed these finding revealing that multiple types of small RNA (FIG. 4) were detected in the samples with the highest percentage (38%) being miRNA. Following miRNA, small nucleolar RNA (snoRNA) resulted in 21% of the population, ribosomal RNA (rRNA) 12% and small nuclear (snRNA) at 10%. Miscellaneous RNA made up 8% of the population, and the remaining 11% were classified as Pseudogenes and Mt-RNAs. These data represent a pool of all cows and are based on mapping criteria.

In total, deep sequencing of the small RNA resulted in 7.5 and 9.2 million reads per sample of which 5-7 million reads mapped to the genome. Following alignment and mapping of the day 17 and 24 samples, there were a total of 214 miRNA that were identified across all groups of which 40 were potential novel miRNA (Table 3). The 214 known and novel miRNA's summarized in Table 3 were identified from a systematic filtering process beginning with a length filter that filtered loci to those with an effective length between 18 and 30 bases. Of the 214 total miRNA identified, the majority (i.e. 129) were found to be represented in all samples, this increased to 166 miRNA in 5 of 6 and 178 miRNA in 4 of 6 groups (Table 3). Very few miRNA were found to be present in only 1 group, 14 miRNA fit this category and of those 8 miRNA were found to be specific to the day 24 pregnant group. However, these specific miRNA were rather low in abundance in these specific samples.

TABLE 2
Gene, GeneBank Number, primer sequence (forward and reverse primer;
5′ to 3′) and location of the primer within the Genebank sequence
for genes amplified during PCR and qPCR.

| | | | | Primer |
|---|---|---|---|---|
| Gene | GeneBank | Primer | Primer Sequence | location |
| Isg15 | 174366 | Forward | 5′-CAGCCAACCAGTGTCTGCAGAGA- | 14-36 |
| | | | 3′ (SEQ ID NO: 4) | |
| | | Reverse | 5′-CCAGGATGGAGATGCAGTTCTGC-3′ | 284-306 |
| | | | (SEQ ID NO: 5) | |
| Mx2 | 173941 | Forward | 5′-CTTCAGAGACGCCTCAGTCG-3′ | 2071-90 |
| | | | (SEQ ID NO: 6) | |
| | | Reverse | 5′-TGAAGCAGCCAGGAATAGTG-3′ | 2283-02 |
| | | | (SEQ ID NO: 7) | |
| Oas1 | 001040606 | Forward | 5′-ACCCTCTCCAGGAATCCAGT-3′ | 1157-76 |
| | | | (SEQ ID NO: 8) | |
| | | Reverse | 5′-GATTCTGGTCCCAGGTCTGA-3′ | 1336-55 |
| | | | (SEQ ID NO: 9) | |
| RPL7 | 001014928 | Forward | 5′-AGGATGGCACGAAAAGCCGGT-3′ | |
| | | | (SEQ ID NO: 10) | |
| | | Reverse | 5′-TCGAACCTTTGGGCTCACACCA-3′ | |
| | | | (SEQ ID NO: 11) | |

TABLE 3
Summary of miRNA sequencing across all treatment
groups at day 17 and 24.

| Control | | EM | | Pregnant | | | |
|---|---|---|---|---|---|---|---|
| day | day | day | day | day | day | # Known | # Novel |
| 17 | 24 | 17 | 24 | 17 | 24 | miRNA | miRNA |
| ✓ | ✓ | ✓ | ✓ | ✓ | ✓ | 116 | 13 |
| ✓ | ✓ | ✓ | ✓ | X | ✓ | 27 | 2 |
| ✓ | X | ✓ | ✓ | X | ✓ | 6 | 0 |
| X | X | X | X | X | ✓ | 4 | 4 |
| X | X | ✓ | X | X | ✓ | 4 | 2 |
| ✓ | X | X | ✓ | X | ✓ | 4 | 0 |
| X | X | X | ✓ | X | X | 2 | 1 |
| ✓ | ✓ | X | X | X | ✓ | 2 | 1 |
| ✓ | ✓ | X | ✓ | X | ✓ | 2 | 1 |
| X | X | X | ✓ | X | ✓ | 2 | 0 |
| ✓ | X | ✓ | ✓ | X | X | 1 | 1 |
| X | X | ✓ | ✓ | X | X | 1 | 0 |
| X | X | ✓ | ✓ | ✓ | ✓ | 1 | 0 |
| ✓ | X | X | ✓ | X | X | 1 | 0 |
| ✓ | X | ✓ | X | X | ✓ | 1 | 0 |
| ✓ | ✓ | ✓ | X | ✓ | ✓ | 0 | 7 |
| X | X | ✓ | X | X | X | 0 | 1 |
| X | ✓ | X | X | X | ✓ | 0 | 1 |
| X | ✓ | X | X | ✓ | X | 0 | 1 |
| X | ✓ | X | ✓ | ✓ | ✓ | 0 | 1 |
| ✓ | X | X | X | X | X | 0 | 1 |
| ✓ | ✓ | X | X | X | X | 0 | 1 |
| ✓ | ✓ | X | ✓ | ✓ | ✓ | 0 | 1 |
| ✓ | ✓ | ✓ | X | X | ✓ | 0 | 1 |

✓ = miRNA were present in that specific group
X = miRNA were not present in that specific group

After setting differential abundance parameters (standardized, i.e. miRNA had to have a cpm greater than or equal to 10 in all 4 replicate samples in at least one of the two samples being compared) for miRNA, we identified 32 differentially expressed loci, representing 27 differentially expressed mature miRNA. The majority (27) of the differentially expressed miRNA were increased in the day 17 EM versus pregnant cows (FIG. 5; Table 4). Specifically, at day 17, of the known miRNA there were 27 miRNA that were significantly greater in abundance in the embryonic mortality group versus pregnant cows and no miRNA were significantly elevated in the remaining groups.

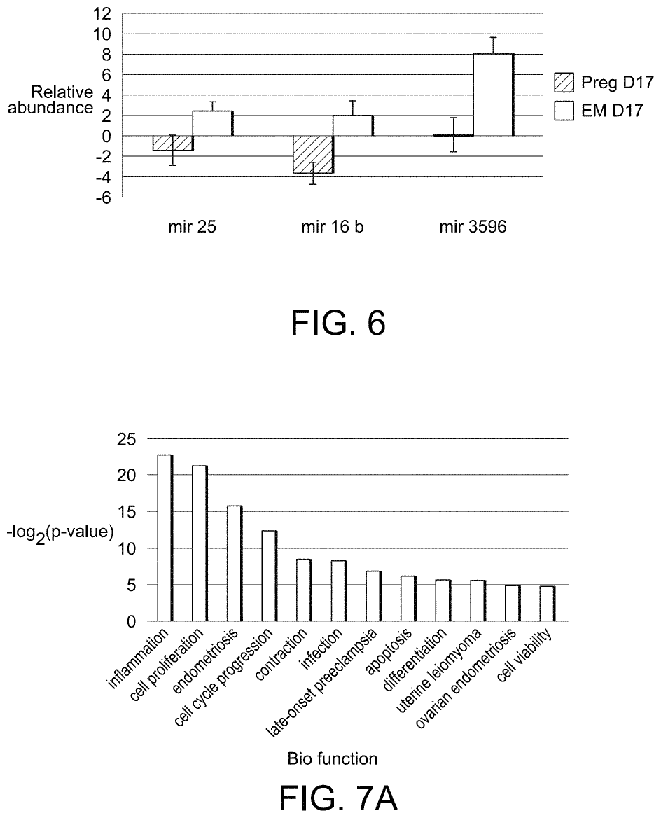

In addition, one novel miRNA that was increased in the day 17 EM versus pregnant cows was also significantly increased in the control compared to the pregnant group (Table 4). On day 24 of gestation, one miRNA was significantly increased between the EM and control group (Table 4), but no other differences were detected. Following sequencing characterization, the differential abundance of 4 mature miRNA were tested using RT-qPCR (miR-16a/b, miR-25, miR-100, miR-3596) at day 17 of gestation. Three of the four miRNA (miR-16a/b, 25, and 3596) were confirmed with RT-qPCR (FIG. 6) to be decreased at day 17 of gestation in the pregnant cows versus those with EM.

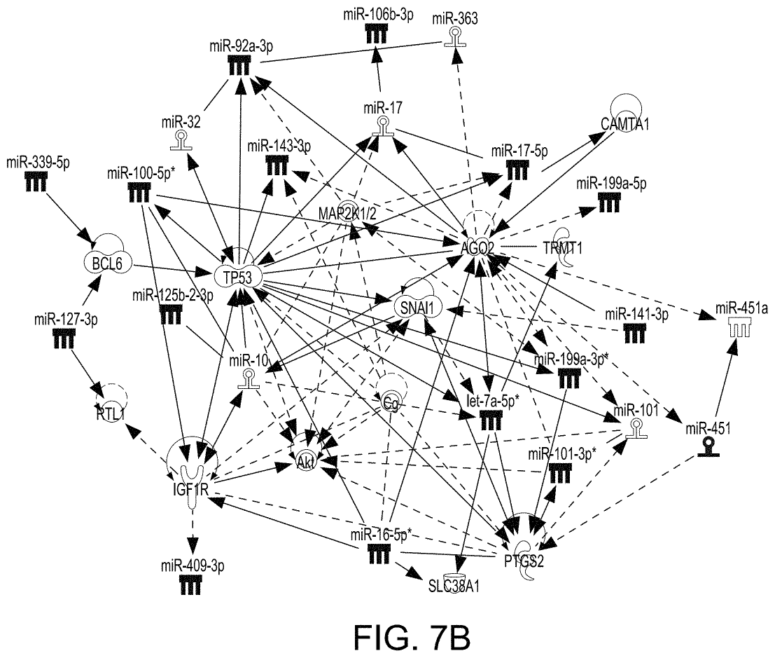

Because the greatest difference existed between the EM and pregnant groups, we focused our Ingenuity Pathway Analysis (IPA) on the 27 known miRNA. MicroRNA that were more abundant in the EM compared to pregnant group were linked to the associated Network functions IPA classification called Cancer, Connective Tissue Disorders, Organismal Injury and Abnormalities, Reproductive System Disease, and Endocrine Disorders as the top 5 Network functions. These included miRNA associated with inflammation, cell proliferation, endometriosis, cell cycle progression, contraction, infection, late-onset preeclampsia, apoptosis, differentiation, uterine leiomyoma, ovarian endometriosis and cell viability (FIG. 7A). Specific gene networks were also identified such as PTGS2, SLC38A1, IGF-1R, Akt, TRMT1, SNAI1, Cg, CAMTA1, MAP2K1/2, BCL6 and TP53 (FIG. 7B).

### Discussion

During early pregnancy, it has been well documented that the bovine embryo begins to elongate into a filamentous conceptus starting about day 15 [33]. This period is also when the conceptus is producing copious amounts of interferon-Tau (IFNT; [34]). The IFNT produced during this period is critical for maintaining or rescuing the CL from regression and extending luteal concentrations of progesterone that are critical for pregnancy establishment [35, 36]. There are many genes regulated by IFNT [33]. Multiple groups have shown that transcripts for ISGs tend to be increased in peripheral leukocytes (white blood cells) by day 16 to 20 of gestation in pregnant compared to non-pregnant cows [12, 13, 37]. Expression of ISG in white blood cells can provide a marker of early pregnancy detection; however, the overall accuracy of these testing platforms are limited by the viral responsive nature of IFNT. In the current study, we chose to only utilize pregnant, non-pregnant and control cows that had specific ISG responses between day 0 and day 17 of gestation. In order to be included in the analysis, pregnant and EM cows had to have an increase in day 17 ISGs compared to day 0, thus suggesting that in both the pregnant and EM animals there was an embryo present at day 17 of gestation capable of secreting IFNT. Similarly, in control animals, ISGs needed to remain low or decreased on day 17 compared to day 0 suggesting that there was no embryo present on day 17 of gestation. We believe that selecting animals based on these specific profiles was key to the results obtained from this experiment and allowed for a more direct comparison on day 17 between the pregnant and EM animals.

Circulating miRNA, which are small non-coding RNAs approximately 22 based pairs in length, have been shown to be accurate biomarkers for a number of human diseases (reviewed by Reid, et al. [38]). Furthermore, specific miRNA have also been detected in serum and plasma collected from pregnant women during gestation [14]. Those that appeared during human pregnancy (e.g. miRNA 512-3p, 517A, 517B, 518B, and 519A) were products of human villous trophoblast that circulate in maternal blood within, or associated with, EVs [23, 39, 40]. EV-associated miRNA, specific to pregnancy, in maternal serum have also been described during gestation in the horse [41]. In addition, miRNA extracted from whole blood have been reported to be different between pregnant and non-pregnant heifers as early as day 16 of gestation; however, whether the preceding miRNA originated from EVs is not known [42]. Burns et al., [43] reported differential abundance of miRNA and protein in microvesicles of uterine flushes between pregnant and cycling ewes on day 14 after. The preceding microvesicles from ovine uterine flushing were not specifically designated as EVs in that study; however, based on their size and protein profile they have potential to be characterized as EVs. A follow up study to the one described above provided evidence that EVs are produced from the trophectoderm and uterine epithelia in the pregnant ewe and are involved in intercellular communication [44].

There were differences in the abundance of circulating EV-derived miRNA between pregnant and EM cows. On day 17 and 24 of gestation, there were a total of 194 and 211 miRNA that successfully tiled to the reference genome. Specifically, there were a notable number of miRNA that were increased in abundance from either control or EM cows when compared to pregnant animals on day 17. Interestingly, Burns et al., [43] reported 27 miRNA specific to cycling (nonpregnant) ewes compared to one unique miRNA in the uterine flushings from pregnant ewes on day 14, which correlates with what we observed in circulation. In addition, there was only 1 pregnancy specific miRNA identified in the uterine flushes from sheep, which was similar to the current study in which we identified none. Interestingly, pregnancy associated glycoproteins (PAG) increase significantly at d 24 of gestation in cattle, thus demonstrating that as early as day 24 it is possible to detect placental products or pregnancy-specific products in maternal circulation [9, 11]. Thus, we hypothesized that at d 24 we would detect pregnancy specific miRNA in circulation; however, we were unable to detect any at d 17 or 24. It is important to point out that novel miRNA for pregnancy or EM detection may actually be down regulated or potentially taken up instead of an increase in abundance. These data also provide evidence that EV-derived miRNA do exist in the circulation of cattle.

The classical model for EV-mediated transfer of miRNA is based on EVs acting as intercellular transfer vehicles of miRNA [17, 45]. A report by Chevillet et al., [46] demonstrated that on average most EVs actually carry less than a single copy of miRNA (0.00825±0.02; miRNA/molecule). If correct, this observation indicates that it would take multiple EVs, with the same miRNA cargo, to influence the biological function of target cells. Several reports of biologically active EVs carrying functional miRNA, mRNA and proteins have been demonstrated in cancer biology and placental biology dealing with viral resistance [47-49]. In the present study, we have not shown any biological relevance of the differentially abundant miRNA. However, if each EV is indeed carrying less than a single miRNA, the large differences in miRNA between the pregnant, EM and control samples is suggestive that these particular miRNA may have biological/functional relevance. The three miRNA (miR-25, -16a/b and 3596) had elevated abundance in the day 17 EM group compared to both the pregnant and control groups. Specifically, miR-25 has been shown to be highly expressed in fetal tissue [50], thus suggesting that miR-25 may be produced by the developing conceptus and secreted into the maternal circulation for specific action. IPA analysis further showed that the specific miRNA increased in the EM compared to the pregnant animals was leading to an upregulation of a pathway involving PTGS2, which is the rate limiting step for prostaglandin production [51]. Thus possibly suggesting that these EM specific miRNA are signaling for an increase in PG production leading to CL regression. Overall, it is evident that the general hypothesis of how EVs are packaged and shuttled throughout the biological systems needs to be closely examined in order to understand EVs mediated transfer of miRNA and its biological significance.

Small RNAs (i.e. miRNA, Piwi-interacting RNAs and small regulatory RNAs) are similar in that they function as regulatory RNAs that are able to direct protein binding to specific nucleotide bases, exert regulation at the transcriptional level, chromatin level or post-transcriptionally [52-54]. Based on RNA profiling, it is clear that the harvested EVs from each experimental group contain a large number of small RNA species (<200 bp) and these profiles seem to be rather consistent across all treatment groups. Again, these data are not surprising based on the evidence in humans that synctiotrophoblast cells produce EVs that contain miRNA that can be found in the maternal circulation [23] along with a similar report from ovine uterine flushings [43]. Based on profiles of microvesicles by Burns et al.[43], as well as this study, it is clear that miRNA are not the sole population of small RNAs present in EVs or microvesicles. Other small RNAs such as piwi-interacting RNAs, small interfering RNAs and repeat-associated RNAs (all <200 bp in length) have been shown to be associated with spermatogenesis and play functional roles in early embryonic development [55, 56]. However, out of all small RNAs discussed in this section, the roles of miRNA in biological systems have been the most clearly defined to this point—especially in regard to epigenetic modification of gene transcription [57-59]. MicroRNA are known to be highly conserved across species and information about the functional role of a miRNA in one species can often be applied to another [60]. Although the present study identified specific miRNA that differed in abundance between pregnant and non-pregnant groups, the exact function and/or source of these specific miRNA remains unclear.

### CONCLUSION

The results of this study support the idea that EV-derived miRNA may provide a useful biomarker for reproduction related fields. Additionally, validation by RT-PCR of the specific miRNA in larger cohorts of animals needs to be completed to determine the robustness of the biomarkers and to move the technology into a high throughput methodology that can used to successful diagnosis early pregnancy. If validation of these specific miRNA allows for detection of individual miRNA differences between pregnant and EM animals, this will allow for significant investigation into in vivo models of pregnancy establishment and embryonic mortality.

While the terms used herein are believed to be well-understood by one of ordinary skill in the art, definitions are set forth to facilitate explanation of certain of the presently-disclosed subject matter.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter. With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, S, C, and/or O” includes A, S, C, and O individually, but also includes any and all combinations and subcombinations of A, S, C, and O.

The foregoing description of preferred embodiments has been presented for purposes of illustration and description. It is not intended to be exhaustive or to limit the invention to the precise form disclosed. Obvious modifications or variations are possible in light of the above teachings. The embodiments were chosen and described to provide the best illustration of the principles of the disclosed subject matter and its practical application to thereby enable one of ordinary skill in the art to utilize the invention in various embodiments and with various modifications as are suited to the particular use contemplated. All such modifications and variations are within the scope of the invention as determined by the claims when interpreted in accordance with the breadth to which they are fairly, legally and equitably entitled.

### REFERENCES

1. Maurer R, Chenault J. Fertilization failure and embryonic mortality in parous and nonparous beef cattle. J. Anim. Sci. 1983; 56:1186-1189.
2. Ahmad N, Schrick F N, Butcher R L, Inskeep E K. Effect of persistent follicles on early embryonic losses in beef cows. Biol. Reprod. 1995; 52:1129-1135.
3. Dunne L, Diskin M, Sreenan J. Embryo and foetal loss in beef heifers between day 14 of gestation and full term. Anim. Reprod. Sci. 2000; 58:39-44.
4. Diskin M G, Morris D G. Embryonic and early foetal losses in cattle and other ruminants. Reprod. Domest. Anim. 2008; 43 Suppl 2:260-267.
5. Diskin M G, Murphy J J, Sreenan J M. Embryo survival in dairy cows managed under pastoral conditions. Anim. Reprod. Sci. 2006; 96:297-311.
6. Wiltbank M C, Baez G M, Garcia-Guerra A, Toledo M Z, Monteiro P L, Melo L F, Ochoa J C, Santos J E, Sartori R. Pivotal periods for pregnancy loss during the first trimester of gestation in lactating dairy cows. Theriogenology 2016; 86:239-253.
7. Fricke P. Scanning the future—Ultrasonography as a reproductive management tool for dairy cattle. J. Dairy Sci. 2002; 85:1918-1926.
8. Perry G A, Smith M F, Lucy M C, Green J A, Parks T E, MacNeil M D, Roberts A J, Geary T W. Relationship between follicle size at insemination and pregnancy success. Proc. Natl. Acad. Sci. USA 2005; 102:5268-5273.
9. Pohler K, Geary T, Johnson C, Atkins J, Jinks E, Busch D, Green J, MacNeil M, Smith M. Circulating bovine pregnancy associated glycoproteins are associated with late embryonic/fetal survival but not ovulatory follicle size in suckled beef cows. J. Anim. Sci. 2013; 91:4158-4167.
10. Pohler K G, Green J A, Geary T W, Peres R F, Pereira M H, Vasconcelos J L, Smith M F. Predicting Embryo Presence and Viability. Adv. Anat. Embryol. Cell Biol. 2015; 216:253-270.
11. Pohler K, Pereira M, Lopes F, Lawrence J, Keisler D, Smith M, Vasconcelos J, Green J. Circulating concentrations of bovine pregnancy-associated glycoproteins and late embryonic mortality in lactating dairy herds. J. Dairy Sci. 2016; 99:1584-1594.
12. Gifford C, Racicot K, Clark D, Austin K, Hansen T, Lucy M, Davies C, Ott T. Regulation of interferon-stimulated genes in peripheral blood leukocytes in pregnant and bred, nonpregnant dairy cows. J. Dairy Sci. 2007; 90:274-280.
13. Green J, Okamura C, Poock S, Lucy M. Measurement of interferon-tau (IFN-τ) stimulated gene expression in blood leukocytes for pregnancy diagnosis within 18-20 d after insemination in dairy cattle. Anim. Reprod. Sci. 2010; 121:24-33.
14. Gilad S, Meiri E, Yogev Y, Benjamin S, Lebanony D, Yerushalmi N, Benjamin H, Kushnir M, Cholakh H, Melamed N. Serum microRNAs are promising novel biomarkers. PLoS One 2008; 3:e3148.
15. Etheridge A, Lee I, Hood L, Galas D, Wang K. Extracellular microRNA: a new source of biomarkers. Mutat. Res. 2011; 717:85-90.
16. Gallo A, Tandon M, Alevizos I, Illei G G. The Majority of MicroRNAs Detectable in Serum and Saliva Is Concentrated in Exosomes. PLoS ONE 2012; 7.
17. Valadi H, Ekstrom K, Bossios A, Sjostrand M, Lee J J, Lotvall J O. Exosome-mediated transfer of mRNAs and microRNAs is a novel mechanism of genetic exchange between cells. Nat. Cell Biol. 2007; 9:654-U672.
18. Gould S J, Raposo G. As we wait: coping with an imperfect nomenclature for extracellular vesicles. J. Extracell. Vesicles 2013; 2.
19. Wang K, Zhang S, Weber J, Baxter D, Galas D J. Export of microRNAs and microRNA-protective protein by mammalian cells. Nucleic Acids Res. 2010:gkq601.
20. Iguchi H, Kosaka N, Ochiya T. Secretory microRNAs as a versatile communication tool. Commun. Integr. Biol. 2010; 3:478-481.
21. Camussi G, Deregibus M C, Bruno S, Cantaluppi V, Biancone L. Exosomes/microvesicles as a mechanism of cell-to-cell communication. Kidney Int. 2010; 78:838-848.
22. Thind A, Wilson C. Exosomal miRNAs as cancer biomarkers and therapeutic targets. J. Extracell. Vesicles 2016; 5.
23. Luo S-S, Ishibashi O, Ishikawa G, Ishikawa T, Katayama A, Mishima T, Takizawa T, Shigihara T, Goto T, Izumi A. Human villous trophoblasts express and secrete placenta-specific microRNAs into maternal circulation via exosomes. Biol. Reprod. 2009; 81:717-729.
24. Théry C, Amigorena S, Raposo G, Clayton A. Isolation and characterization of exosomes from cell culture supernatants and biological fluids. Curr. Protoc. Cell Biol. 2006:3.22. 21-23.22. 29.
25. Hung M E, Leonard J N. Stabilization of exosome-targeting peptides via engineered glycosylation. J. Biol. Chem. 2015; 290:8166-8172.
26. Mathivanan S, Lim J W, Tauro B J, Ji H, Moritz R L, Simpson R J. Proteomics analysis of A33 immunoaffinity-purified exosomes released from the human colon tumor cell line LIM1215 reveals a tissue-specific protein signature. Mol. Cell. Proteomics 2010; 9:197-208.
27. Navakanitworakul R, Hung W-T, Gunewardena S, Davis J S, Chotigeat W, Christenson L K. Characterization and Small RNA Content of Extracellular Vesicles in Follicular Fluid of Developing Bovine Antral Follicles. Sci. Rep. 2016; 6.
28. Langmead B, Salzberg S L. Fast gapped-read alignment with Bowtie 2. Nat Methods 2012; 9:357-359.
29. Jiang P, Wu H, Wang W, Ma W, Sun X, Lu Z. MiPred: classification of real and pseudo microRNA precursors using random forest prediction model with combined features. Nucleic Acids Res. 2007; 35:W339-344.
30. Robinson M D, McCarthy D J, Smyth G K. edgeR: a Bioconductor package for differential expression analysis of digital gene expression data. Bioinformatics 2010; 26:139-140.
31. Benjamini Y, Hochberg Y. Controlling the false discovery rate: a practical and powerful approach to multiple testing. Journal of the Royal Statistical Society. Series B (Methodological) 1995:289-300.
32. Kirby C J, Wilson S J, Lucy M C. Response of dairy cows treated with bovine somatotropin to a luteolytic dose of prostaglandin F 2α. J. Dairy Sci. 1997; 80:286-294.
33. Spencer T E, Sandra O, Wolf E. Genes involved in conceptus-endometrial interactions in ruminants: insights from reductionism and thoughts on holistic approaches. Reproduction 2008; 135:165-179.
34. Robinson R S, Hammond A J, Wathes D C, Hunter M G, Mann G E. Corpus luteum-endometrium-embryo interactions in the dairy cow: Underlying mechanisms and clinical relevance. Reprod. Domest. Anim. 2008; 43:104-112.
35. Godkin J, Bazer F, Thatcher W, Roberts R. Proteins released by cultured day 15-16 conceptuses prolong luteal maintenance when introduced into the uterine lumen of cyclic ewes. J. Reprod. Fertil. 1984; 71:57-64.
36. Roberts R, Imakawa K, Niwano Y, Kazemi M, Malathy P-V, Hansen T, Glass A, Kronenberg L. Interferon production by the preimplantation sheep embryo. J. Interferon Res. 1989; 9:175-187.
37. Stevenson J, Dalton J, Ott T, Racicot K, Chebel R. Correlation between reproductive status and steady-state messenger ribonucleic acid levels of the resistance gene, MX2, in peripheral blood leukocytes of dairy heifers. J. Anim. Sci. 2007; 85:2163-2172.
38. Reid G, Kirschner M B, van Zandwijk N. Circulating microRNAs: Association with disease and potential use as biomarkers. Crit. Rev. Oncol. Hematol. 2011; 80:193-208.
39. Miura K, Miura S, Yamasaki K, Higashijima A, Kinoshita A, Yoshiura K-i, Masuzaki H. Identification of pregnancy-associated microRNAs in maternal plasma. Clin. Chem. 2010; 56:1767-1771.
40. Kotlabova K, Doucha J, Hromadnikova I. Placental-specific microRNA in maternal circulation-identification of appropriate pregnancy-associated microRNAs with diagnostic potential. J. Reprod. Immunol. 2011; 89:185-191.
41. Cameron A, da Silveira J, Bouma G, Bruemmer J. Evaluation of exosomes containing miRNA as an indicator of pregnancy status in the mare. J. Equine Vet. Sci. 2011; 31:314-315.
42. Ioannidis J, Donadeu F X. Circulating miRNA signatures of early pregnancy in cattle. BMC Genomics 2016; 17:1.
43. Burns G, Brooks K, Wildung M, Navakanitworakul R, Christenson L K, Spencer T E. Extracellular Vesicles in Luminal Fluid of the Ovine Uterus. PLoS One. 2014; 9:1-11.
44. Burns G W, Brooks K E, Spencer T E. Extracellular Vesicles Originate from the Conceptus and Uterus During Early Pregnancy in Sheep. Biol. Reprod. 2016:biolreprod. 115.134973.
45. Raposo G, Stoorvogel W. Extracellular vesicles: exosomes, microvesicles, and friends. J. Cell Biol. 2013; 200:373-383.
46. Chevillet J R, Kang Q, Ruf I K, Briggs H A, Vojtech L N, Hughes S M, Cheng H H, Arroyo J D, Meredith E K, Gallichotte E N. Quantitative and stoichiometric analysis of the microRNA content of exosomes. Proc. Natl. Acad. Sci. USA 2014; 111:14888-14893.
47. Skog J, Würdinger T, van Rijn S, Meijer D H, Gainche L, Curry W T, Carter B S, Krichevsky A M, Breakefield X O. Glioblastoma microvesicles transport RNA and proteins that promote tumour growth and provide diagnostic biomarkers. Nat. Cell Biol. 2008; 10:1470-1476.
48. Delorme-Axford E, Donker R B, Mouillet J-F, Chu T, Bayer A, Ouyang Y, Wang T, Stolz D B, Sarkar S N, Morelli A E. Human placental trophoblasts confer viral resistance to recipient cells. Proc. Natl. Acad. Sci. USA 2013; 110:12048-12053.
49. Morello M, Minciacchi V, de Candia P, Yang J, Posadas E, Kim H, Griffiths D, Bhowmick N, Chung L, Gandellini P. Large oncosomes mediate intercellular transfer of functional microRNA. Cell cycle 2013; 12:3526-3536.
50. Coutinho L L, Matukumalli L K, Sonstegard T S, Van Tassell C P, Gasbarre L C, Capuco A V, Smith T P. Discovery and profiling of bovine microRNAs from immune-related and embryonic tissues. Physiol. Genomics 2007; 29:35-43.
51. Bazer F W. Pregnancy recognition signaling mechanisms in ruminants and pigs. J. Anim. Sci. Biotechnol. 2013; 4:1.
52. Carthew R W. A new RNA dimension to genome control. Science 2006; 313:305-306.
53. Pillai R S. MicroRNA function: multiple mechanisms for a tiny RNA? RNA 2005; 11:1753-1761.
54. Ambros V, Chen X. The regulation of genes and genomes by small RNAs. Development 2007; 134:1635-1641.
55. He Z, Kokkinaki M, Pant D, Gallicano G I, Dym M. Small RNA molecules in the regulation of spermatogenesis. Reproduction 2009; 137:901-911.
56. Krawetz S A, Kruger A, Lalancette C, Tagett R, Anton E, Draghici S, Diamond M P. A survey of small RNAs in human sperm. Hum. Reprod. 2011:der329.
57. Kim D H, Setrom P, Snøve O, Rossi J J. MicroRNA-directed transcriptional gene silencing in mammalian cells. Proc. Natl. Acad. Sci. USA 2008; 105:16230-16235.
58. Valeri N, Vannini I, Fanini F, Calore F, Adair B, Fabbri M. Epigenetics, miRNAs, and human cancer: a new chapter in human gene regulation. Mamm. Genome 2009; 20:573-580.
59. Khraiwesh B, Arif M A, Seumel G I, Ossowski S, Weigel D, Reski R, Frank W. Transcriptional control of gene expression by microRNAs. Cell 2010; 140:111-122.
60. Massirer K B, Pasquinelli A E. The evolving role of microRNAs in animal gene expression. Bioessays 2006; 28:449-452.

What is claimed:

1. A method for determining early embryonic mortality (EM) in a female bovine, comprising:
isolating serum from a blood sample obtained from the female bovine at from about 15 to about 30 days of gestation;
determining an extracellular vesicle derived micro-ribonucleic acid expression profile of the serum (serum EV miRNA);
comparing the serum EV miRNA expression profile to at least one reference serum EV miRNA expression profile;
by the comparing, determining a serum EV miRNA expression profile indicative of early EM (EM EV miRNA expression profile) in the female bovine; and
placing the female bovine in a rebreeding or resynchronization protocol.

2. The method of claim 1, including determining the at least one reference serum EV miRNA expression profile from one or both of: (i) a blood sample obtained from one or more reference pregnant female bovines at from about 15 to about 30 days of gestation, and (ii) a blood sample obtained from one or more reference non-pregnant female bovines.

3. The method of claim 2, including providing the at least one reference serum EV miRNA expression profile from the blood sample obtained at from about 15 to about 30 days of gestation from the reference pregnant female bovine.

4. The method of claim 2, further including isolating EVs from said serum to provide a sample of isolated EVs.

5. The method of claim 4, further including extracting ribonucleic acids from said sample of isolated EVs and amplifying and quantifying the EV miRNA by high-throughput sequencing and reverse transcriptase quantitative PCR (RT-qPCR) to provide the serum EV miRNA profile and the at least one reference serum EV miRNA profile.

6. The method of claim 5, including determining the EM EV miRNA expression profile consisting of an increased amount of at least one of miR-25, miR-16a/b, or miR-3596 compared to the at least one reference serum EV miRNA expression profile.

7. The method of claim 5, including determining the EM EV miRNA expression profile consisting of an increased amount of miR-25, miR-16a/b, and miR-3596 compared to the at least one reference serum EV miRNA expression profile.

8. The method of claim 6, including amplifying the EV miRNA using primers selected from the group consisting of SEQ ID NO:1, SEQ ID NO:2, and SEQ ID NO:3.

9. The method of claim 7, including amplifying the EV miRNA using primers selected from the group consisting of SEQ ID NO:1, SEQ ID NO:2, and SEQ ID NO:3.

10. The method of claim 6, including standardizing the EM EV miRNA expression profile and the at least one reference serum EV miRNA expression profile by normalizing effective miRNA read counts to a number of counts per million reads (cpm) and retaining only loci with a cpm greater than or equal to 10.

11. The method of claim 7, including standardizing the EM EV miRNA expression profile and the at least one reference serum EV miRNA expression profile by normalizing to a number of effective miRNA read counts per million reads (cpm) and retaining only loci with a cpm greater than or equal to 10.

12. An extracellular vesicle derived micro-ribonucleic acid expression profile indicative of embryonic mortality (EM EV miRNA expression profile) at from about 15 to about 30 days of gestation in a female bovine, comprising an increased amount of miR-25, miR-16a/b, and miR-3596 in a serum EV miRNA expression profile of the female bovine compared to an amount of miR-25, miR-16a/b, and miR-3596 in at least one reference serum EV miRNA expression profile.

13. The expression profile of claim 12, wherein the amounts of miR-25, miR-16a/b, and miR-3596 are determined by high-throughput sequencing and reverse transcriptase quantitative PCR (RT-qPCR).

14. The expression profile of claim 13, wherein miR-25, miR-16a/b, and miR-3596 are amplified using primers selected from the group consisting of SEQ ID NO:1, SEQ ID NO:2, and SEQ ID NO:3.

15. The expression profile of claim 12, wherein the at least one reference serum EV miRNA expression profile is obtained from one or both of: (i) a blood sample obtained from one or more reference pregnant female bovines at from about 15 to about 30 days of gestation, and (ii) a blood sample obtained from one or more reference non-pregnant female bovines.

16. The expression profile of claim 12, wherein the at least one reference serum EV miRNA expression profile is obtained from a blood sample obtained from one or more reference pregnant female bovines at from about 15 to about 30 days of gestation.

17. The expression profile of claim 12, wherein the EM EV miRNA expression profile and the at least one reference serum EV miRNA expression profile are standardized by normalizing to a number of effective miRNA read counts per million reads (cpm) and retaining only loci with a cpm greater than or equal to 10.

18. A kit for determining early embryonic mortality (EM) in a female bovine, comprising:
a primer array comprising primer sequences for determining expression levels of one or more extracellular vesicle micro-ribonucleic acids (EV miRNAs) indicative of said EM;
optionally, reagents for extracting the EV miRNAs; and
optionally, reagents for performing reverse transcriptase quantitative PCR (RT-qPCR) of the extracted EV miRNA.

19. The kit of claim 18, wherein the primer array comprises primer sequences for determining expression levels of one or more of miR-25, miR-16a/b, and miR-3596 extracted from an extracellular vesicle micro-RNA (EV miRNA).

20. The kit of claim 19, wherein the primer array comprises the primer sequences set forth as SEQ ID NO:1, SEQ ID NO:2, and SEQ ID NO:3.

21. The kit of claim 19, wherein the primer array consists of the primer sequences set forth as SEQ ID NO:1, SEQ ID NO:2, and SEQ ID NO:3.


SEQUENCE LISTING (11 sequences)

SEQ ID NO: 1 - DNA, 21 bp, artificial sequence
  miR-16/ab Primer (5′ - 3′)
uagcagcacguaaauauuggc

SEQ ID NO: 2 - DNA, 22 bp, artificial sequence
  miR-25 Primer (5′ - 3′)
cauugcacuugucucggucuga

SEQ ID NO: 3 - DNA, 22 bp, artificial sequence
  miR-3596 Primer (5′ - 3′)
aaccacacaaccuacuaccuca

SEQ ID NO: 4 - DNA, 23 bp, artificial sequence
  Isg15 Forward Primer (5′ - 3′)
cagccaaccagtgtctgcagaga

SEQ ID NO: 5 - DNA, 23 bp, artificial sequence
  Isg15 Reverse Primer (5′ - 3′)
ccaggatggagatgcagttctgc

SEQ ID NO: 6 - DNA, 20 bp, artificial sequence
  Mx2 Forward Primer (5′ - 3′)
cttcagagacgcctcagtcg

SEQ ID NO: 7 - DNA, 20 bp, artificial sequence
  Mx2 Reverse Primer (5′ - 3′)
tgaagcagccaggaatagtg

SEQ ID NO: 8 - DNA, 20 bp, artificial sequence
  Oas1 Forward Primer (5′ - 3′)
accctctccaggaatccagt

SEQ ID NO: 9 - DNA, 20 bp, artificial sequence
  Oas1 Reverse Primer (5′ - 3′)
gattctggtcccaggtctga

SEQ ID NO: 10 - DNA, 21 bp, artificial sequence
  RPL7 Forward Primer (5′ - 3′)
aggatggcacgaaaagccggt

SEQ ID NO: 11 - DNA, 22 bp, artificial sequence
  RPL7 Reverse Primer (5′ - 3′)
tcgaacctttgggctcacacca